(19) United States
(12) Patent Application Publication
(10) Pub. No.: US 2007/0016297 A1
(43) Pub. Date: Jan. 18, 2007

(54) Prostheses for Spine Facets

(75) Inventor: Wesley M. Johnson, Tampa, FL (US)

Correspondence Address: SMITH HOPEN, PA, 180 PINE AVENUE NORTH, OLDSMAR, FL, 34677, US

(73) Assignee: UNIVERSITY OF SOUTH FLORIDA, Tampa, FL (US)

(21) Appl. No.: 11/419,085

(22) Filed: May 18, 2006

Related U.S. Application Data
(63) Continuation of application No. PCT/US04/38753, filed on Nov. 18, 2004.

(51) Int. Cl. A61F 2/44

(52) U.S. Cl. 623/17.11

(57) ABSTRACT

In accordance with the present invention is provided a prosthesis for the replacement of at least a portion of the bone of a facet located on a host mammalian vertebra. The prosthesis including a posterior arch structure having a plurality of bearing attachment surfaces, a plurality of facet bearing buttons releasably engaged with the bearing attachment surfaces, and a plurality of bioresorbable attachment screws to secure the posterior arch structure to the posterior vertebral articular process of the mammalian vertebra.

### CROSS REFERENCE TO RELATED APPLICATION

This application is a Continuation of International Application No. PCT/US04/38753 filed on Nov. 18, 2004 which, claims priority to U.S. Provisional Patent Application No. 60/520,963 filed by the same inventor on Nov. 18, 2003, entitled: “Prostheses for Lumbar Spine Facets”.

### BACKGROUND OF THE INVENTION

The end result of lumbar degenerative spondylosis comes from a combination of normal aging, disc dehydration, annulus disruption, and inflammation. The discs become desiccated, leaving the lumbar facets to carry more weight than for which they are suited. Ultimately, this leads to facet hypertrophy and spinal canal and foraminal compromise. The current operative treatment of lumbar spondylosis involves some combination of posterior decompression by laminotomy or laminectomy, with or without facetectomy, removal of the degenerated disc, with or without fusion via an anterior technique, posterior technique, or both.

This approach, while often successful, is not ideal. Fusing one segment leads to increased stresses on adjacent motion segments, which will accelerate the degenerative process in those locations. The long-term effects of indwelling metallic instrumentation, while poorly documented, include stress-shielding, infection, a risk of hardware fatigue, breakage, migration, particulate debris which can cause inflammation, and radiographic artifacts.

To avoid the problems of permanent implantation and fusion, efforts have been made to replace the natural disc with artificial discs of various designs. There is a significant population of younger patients in whom cervical degenerative disc disease is the only cause of pain, without any facet degeneration. In these patients, disc replacement alone is a viable solution. In the lumbar spine, only a small number of patients have isolated disc disease, without facet arthropathy. In this population, successful disc replacement does not completely address the disease process, as the degenerated joints are still a problem as long as they are still subject to motion, and as long as spinal stenosis, instability, etc. remain.

The current surgical strategy known in the art is to remove the facets or to fuse the vertebra. Fusing the vertebra puts additional stress on the adjacent discs, which can result in early failure, and limits patient flexibility. The existence of an effective facet replacement and disc prostheses together would radically change the surgical strategy eliminating lumbar fusions altogether. To completely address the disease process, both disc and facet replacement would be required.

Another approach known in the art is prosthetic fixation. Prosthetic fixation involves permanent screw fixation of a prostheses to the host vertebral body in the vicinity of the pedicles. Such permanent screws can migrate causing serious complications.

Yet another approach in the art is to provide surface treatments of the prostheses, where it contacts the host living bone, using osteoconductive and/or osteoinductive substances. The surface areas, so treated, are small and true incorporation of the prostheses is not possible. Without incorporation, the prostheses may loosen with time and migrate causing serious complications.

Additional prosthetic devices known in the art are overly complex and commonly require mechanical fixation to existing bone in the posterior aspect of the vertebral body. In addition to these devices not addressing diseases of the lamina and its complex structure, the complexity of these devices commonly requires many permanent screws and connectors rendering them impractical. The fixations may loosen with time and cause serious complications.

Additionally, none of the prosthetic devices known in the art address the range of facet angles and vertebral body sizes that exist. Devices known in the art do not describe a device or method capable of accommodating these differences between host vertebras.

Thus it can be seen that previous approaches to provide a prostheses that replaces the vertebral posterior structure including the facets are inadequate. It is therefore useful to have a novel prosthesis that can achieve true volumetric bioincorporation without the aforementioned shortcomings.

Accordingly, what is needed in the art is a spinal facet replacement device and procedure that would allow for posterior decompression, restabilization, and preservation of motion with a mechanically simple device that is easily adaptable to various facet angles and vertebral body sizes.

However, in view of the prior art considered as a whole at the time the present invention was made, it was not obvious to those of ordinary skill in the pertinent art how the identified need could be fulfilled.

### SUMMARY OF THE INVENTION

The device of the present invention allows for the repair of diseased or damaged posterior spine elements in the spine, including, but not limited to the lumbar region. The device returns the mechanical function of the facets to normal. The invention allows the surgeon to select from a range of standard components to configure the device for individual patients. The device is generally used in concert with a disc prostheses but need not be for normal or near normal disc heights.

The present invention is a human made replacement for the back parts of the spine, including a standard block of ceramic or other biocompatible material, fitted with small plastic buttons for wear, attached to the host vertebra with temporary screws. The host body absorbs the screws over time. Bone from the vertebra grows into the block making it part of the body. As such, the present invention replicates the facet motion with a type of sliding joint, which also functions to limit mechanical motion. Implantation of the device includes a specific placement and rotation maneuver to position the device for engagement with the host vertebral body and the facets of other vertebra. Other facets could be natural or prosthetic.

In accordance with the present invention, a prosthesis for the replacement of at least a portion of the bone of a facet located on a host mammalian vertebra is provided. The prosthesis includes a posterior arch structure having a plurality of bearing attachment surfaces, a plurality of facet bearing buttons releasably engaged with the bearing attachment surfaces and a plurality of bioresorbable attachment screws to secure the posterior arch structure to the posterior vertebral articular process of the mammalian vertebra.

In an additional embodiment, the prosthesis includes least one pedicle extending from the posterior arch structure. The pedicles are capable of mating at least one surgically prepared fenestra of the mammalian vertebra. The pedicles are adapted to receive a bioresorbable attachment screws to further secure the prosthesis to the host. In a preferred embodiment, there are two cylindrically shaped pedicles and two associated attachment screws. However, it is within the scope of the present invention to have pedicles of various dimension and shapes as required by the implantation procedure of the prosthesis.

In addition to the pedicles, the prosthesis in accordance with the present invention may include a posterior protrusion in a shape approximating a spinous process. This posterior protrusion is capable of resection with a bone piece originating from the spinous process of the host. The posterior protrusion may be bioincorporable with the host and further include a plurality of volumetric voids to establish volumetric porosity of the protrusion. In a particular embodiment, the volumetric porosity is established utilizing voids exhibiting diameters between 75 μm and 500 μm. In an additional embodiment, the voids may be either internally or externally coated with a substance to enhance bioincorporation of the protrusion. These coatings may include osteoinductive material, such as hydroxyapitite, or osteoconductive material, such as bone morphogenic protein, or other bioincorporation enhancing substances as are commonly known in the art.

The prosthesis in accordance with the present invention includes a plurality of bearing attachment surfaces further comprises. The number and location of the bearing attachment surfaces in dependent upon the replacement needs of the host. In a particular embodiment, the prosthesis includes at least one superior prosthetic facet to contact an inferior facet of a superior vertebral body of the vertebra, the at least one superior prosthetic facet to receive at least one of the plurality of facet bearing buttons and, at least one inferior prosthetic facet to contact a superior facet of an inferior vertebral body of the vertebra, the at least one inferior prosthetic facet to receive at least one of the plurality of facet bearing buttons. In a specific embodiment, the prosthesis includes two superior prosthetic facets and two inferior prosthetic facets.

It is within the scope of the invention to fabricate the prosthesis from a variety of biocompatible materials, including, but not limited to, ceramic, glass reinforced polymer and carbon reinforced polymer. The prosthesis may additional be enhanced to provide bioincorporation of the posterior arch structure. Accordingly, the posterior arch structure may be fabricated to include a plurality of volumetric voids to establish volumetric porosity of the posterior arch structure. In a particular embodiment, the voids are between about 75 μm and 500 μm in diameter. Additionally, the voids may be internally or externally coated with a material to increase bioincorporation. The coating may include an osteoinductive material, such as hydroxyapitite, or an osteoconductive material, such as bone morphogenic protein. Additional bioincorporation enhancing materials are also within the scope of the present invention.

The bearing attachment surfaces of the posterior arch structure are substantially planar and positioned at a predetermined angle with respect to a sagittal plane of the host. As such, the superior bearing attachment surfaces are positioned to face substantially medially relative to the posterior arch structure, and the inferior bearing attachment surfaces are positioned to face substantially laterally relative to the posterior arch structure. The bearing attachment surfaces are fitted with a plurality of facet bearing buttons. The buttons may be fabricated from an ultra-high molecular weight polyethylene. The buttons further include a button cap and a button stem. The button cap may be shaped to be substantially concave, substantially convex or substantially flat. The button stem may further include a locking barb to secure the button within an aperture of the bearing attachment surface.

In a particular embodiment in accordance with the present invention, a prosthesis for the replacement of at least a portion of the bone of a facet located on a host mammalian vertebra in provided, including, a posterior arch structure having two superior bearing attachment surfaces and two inferior bearing attachment surfaces, two pedicles extending from the posterior arch structure, the two pedicles dimensioned to engage with two fenestra provided in the host vertebra, two superior facet bearing buttons releasably engaged with the two superior bearing attachment surfaces, two inferior facet bearing buttons releasably engaged with the two inferior bearing attachment surfaces and two bioresorbable attachment screws to secure the posterior arch structure to the posterior vertebral articular process of the mammalian vertebra through the two pedicles.

The prosthesis in accordance with the present invention provides a customizable solution for spine facet replacement procedures. In accordance with the present invention, a kit for use in surgical replacement of a spine facet would include a plurality of posterior arch structures in accordance with the present invention in a plurality of sizes and facet angles, a plurality of facet bearing buttons, and a plurality of bioresorbable attachment screws. In practice the surgeon will select the appropriate size posterior arch structure block and facet bearing buttons to accommodate the patient specific needs. The selection will be made based on simple linear measurements made of the intact facets on the superior and inferior vertebrae. The various sizes and facet angles provided by this device would allow for a customizable solution for spine replacement currently unavailable in the state of the art.

A method of using the prostheses in accordance with the present invention includes, introducing the prostheses into a host during implantation further including placing a superior end of the prostheses with a posterior arch structure stems facing caudally, rotating about the lateral axis of the posterior arch structure, such that the prosthetic posterior arch structure stems line up with the prepared holes and the superior prosthetic facets contacting the inferior facets of the superior vertebral body, and inserting prosthetic arch structure stems into the prepared holes.

The present invention is a human made replacement for the back parts of the spine, lower spine, or lumbar region. A standard block of ceramic or other biocompatible material is fitted with small plastic buttons for wear and attached to the vertebra with temporary screws. The body absorbs the screws over time. Bone from the vertebra grow into the block making them part of the body. The present invention would replicate the facet motion with a type of sliding joint, which also functions to limit mechanical motion. Implantation of the device includes a specific placement and rotation maneuver to engage with the host vertebral body and the facets of other vertebra.

### BRIEF DESCRIPTION OF THE DRAWINGS

For a fuller understanding of the nature and objects of the invention, reference should be made to the following detailed description, taken in connection with the accompanying drawings, in which:

FIG. 1 is a diagrammatic view of the monolithic posterior arch structure, facet bearing button and pedical screws in accordance with the present invention;

FIG. 2 is a diagrammatic posterior view of the monolithic posterior arch structure, facet bearing button and pedical screws in accordance with the present invention;

FIG. 3 is an alternate diagrammatic view of the monolithic posterior arch structure in accordance with the present invention;

FIG. 4 is a diagrammatic side view of the prothesis in accordance with the present invention;

FIG. 5 is a diagrammatic top-down view of the prosthesis in accordance with the present invention;

FIG. 6 is an alternate diagrammatic top-down view of the prosthesis in accordance with the present invention;

FIG. 7 is a diagrammatic view of the prosthesis in accordance with the present invention, detaling the facet bearing mounting holes;

FIG. 8 is a diagrammatic dual view of the prosthesis in accordance with the present invention;

FIG. 9 is a diagrammatic dual view of the prosthesis in accordance with the present invention;

FIG. 10 is a diagrammatic dual view of the prosthesis in accordance with the present invention;

FIG. 11 is a diagrammatic view of the prosthesis in accordance with the present invention detailing the attachment mechanism for the spinous process;

FIG. 12 is a diagrammatic view of the facet bearing button in accordance with the present invention having a substantially concave contour;

FIG. 13 is a diagrammatic view of the facet bearing button in accordance with the present invention having a substantially convex contour;

FIG. 14 is a diagrammatic view of the facet bearing button in accordance with the present invention having a substantially flat contour; and

FIG. 15 is a diagrammatic view of the fenestrated pedicle attachment screw in accordance with the present invention.

### DETAILED DESCRIPTION OF THE PREFERRED EMBODIMENT

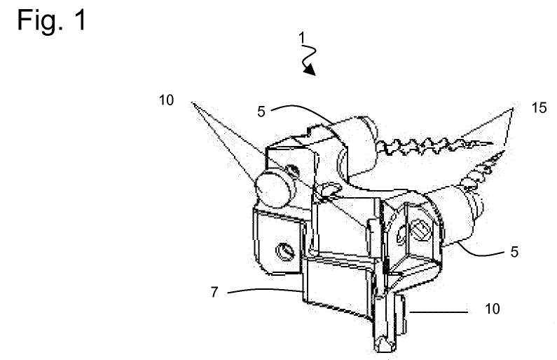

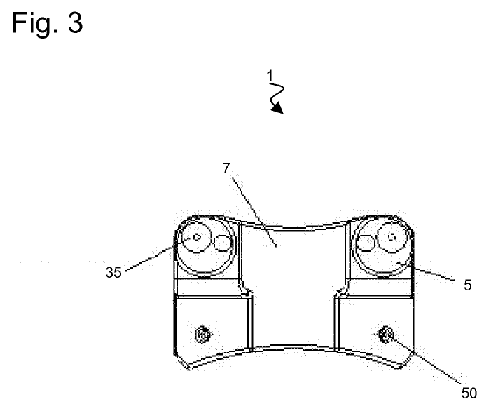

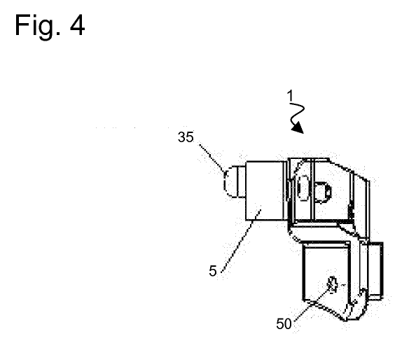

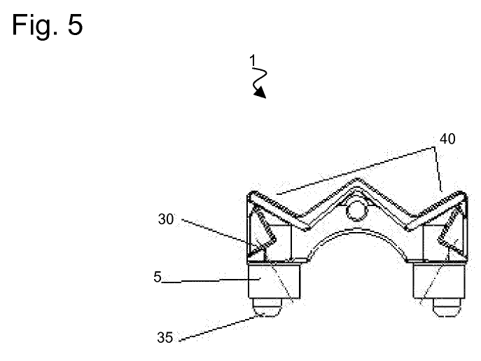

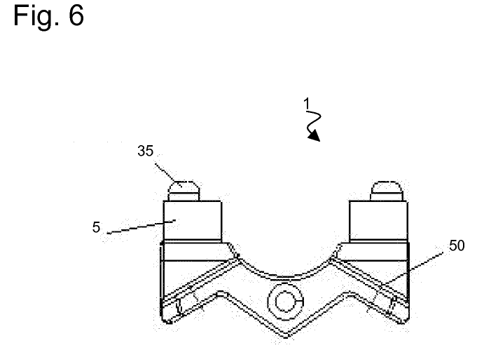

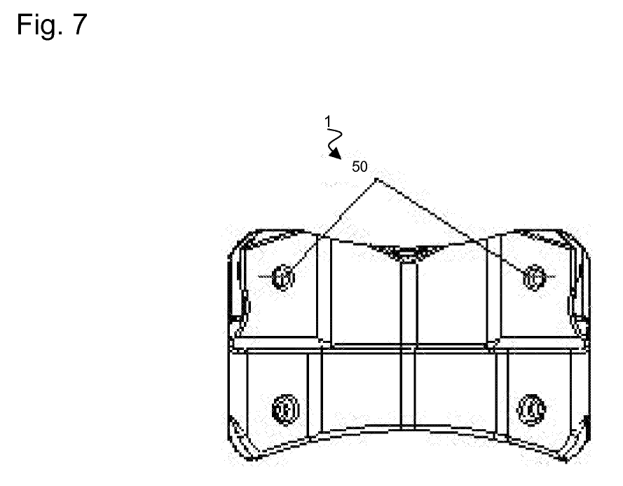

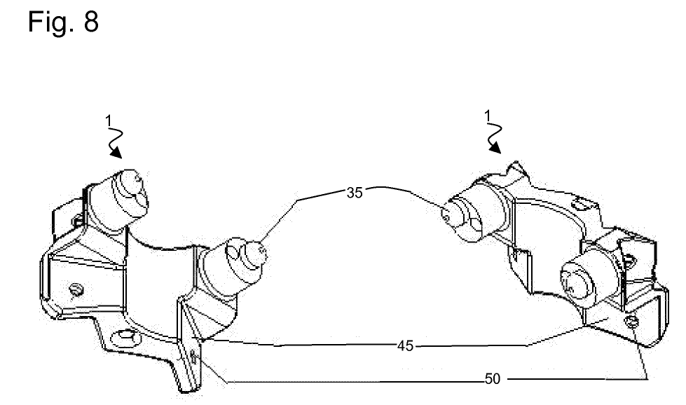

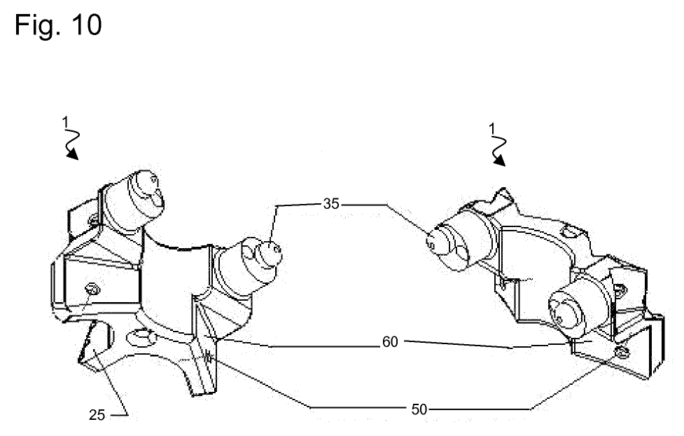

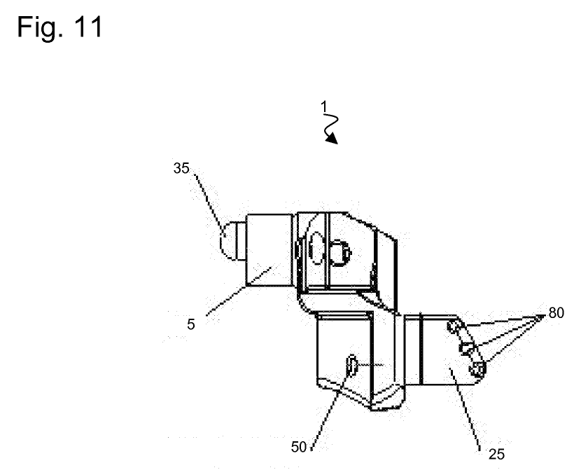

With reference to FIG. 1 through FIG. 12, the present invention comprises a combination of various sizes of three components configured into a prosthesis 1 for the replacement of a spinal facet. The first part is a monolithic block referred to as a posterior arch structure 7. The second part is a bearing element called the facet bearing button 10, 15. The third part is the attachment screw set 20. In a particular embodiment, these parts are arranged in a surgical kit which allows the surgeon a range of different combinations of posterior arch structure components, bearing buttons, and screw lengths to fit a wide range of individual patients.

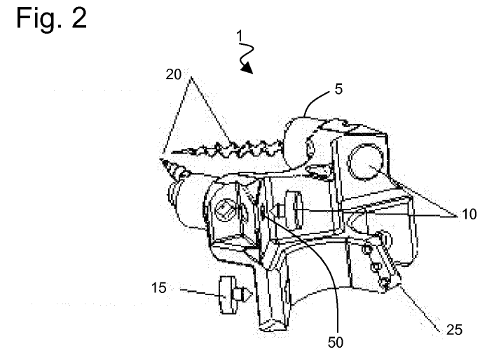

In accordance with the present invention, two different median posterior arch structure (PAS) block sizes are provided. One for L1, L2, and L3 (designated PAS123) and one for L4 and L5 (designated PAS45). In both cases the PAS block is constructed of a similar material utilizing a similar processes. The major difference between PAS123 and PAS45 is the angle of the facet bearing button mounting surfaces. The PAS blocks are comprised of fully or partially fired ceramic or other material known to the art, such as glass or carbon reinforced polymer or other polymer, that has been machined or cast to dimensions and configuration approximating the posterior elements of a vertebra or lumbar vertebra, as shown in FIGS. 1 and 2.

In a particular embodiment, the posterior arch structure includes two pedicles 5, or protruding stems, for mating with the host vertebra and a posterior protrusion 25 resembling the spinous process. As such, through a surgical procedure, the host vertebral body, in the vicinity of the pedicles, is bored out, or otherwise formed, using such techniques as broaching, to provide two cavities or holes. In the preferred embodiment the stems are cylindrical but may be rectangular or any other shape. The depth of the cavities is not expected to exceed the surface of the vertebral body and are intended to match the depth of the posterior arch structure pedicles 25. Additionally, the shape of the very end of the posterior arch strucuture stems are to be conformal with the shape of the formed mating cavity or hole. The posterior protrusion 25, in a shape approximating the spinous process, functions as a strengthening web for the arch structure and as an attachment point for a resected bone piece from the original spinous process. The transverse processes of a natural vertebral body are not approximated.

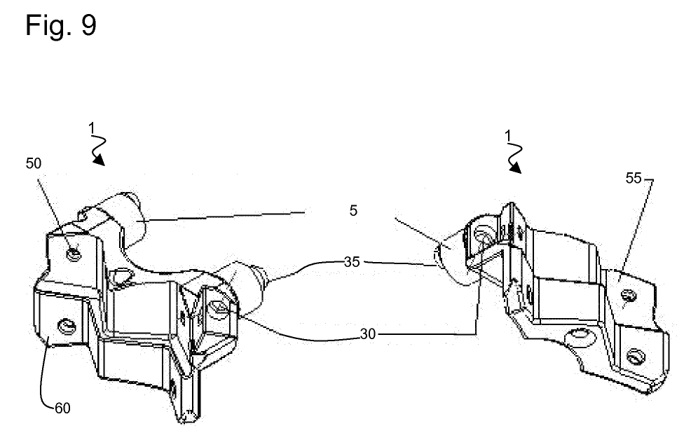

The posterior arch structure pedicles, or stems, may be prepared to contain a range of volumetrically connected voids and porosity in an approximate range of 70 μm to 500 μm diameter and various depths, through selective heating, etching, and/or machining, or other method, or manufacturing means known in the art. The selected volumetric porosity, may be coated, internally and/or externally with osteoconductive substance(s), such as hydroxyapitite and/or osteoinductive substance(s) such as bone morphogenic proteins (BMPs) to produce bone in-growth and ultimate bioincorporation of the device. The PAS stems also have holes 30, as illustrated in FIG. 9, to receive the fenestrated attachment screws. The ends of the stems 35 are formed conformal with the mating cavities or holes to enhance bone in-growth and are shaped to minimize the entrapment, impingement, impaction, or other contact with the nerve roots during implantation.

The spinous process region of the prostheses 25 is formed to provide volumetric porosity in the range of 70 μm to 500 μm using the same techniques as employed for the PAS stems. This region of the prostheses may be coated, internally and/or externally with an osteoconductive substance, such as hydroxyapitite and/or osteoinductive substance such as bone morphogenic proteins (BMPs) to produce bone in-growth from the original spinous process bone piece to the device. The protrusion may contain at least one notch 80 for attachment, by means such as suture or staple or other means known in the art, of the resected bone piece which itself is attached to the supraspinous ligament, to the PAS spinous process region.

With reference to FIG. 9, the mounting surfaces for the facet bearing buttons are substantially flat and at a predetermined angle with respect to the sagittal plane of the host vertebra. For PAS123 block the angles are 30° for the superior surfaces 55 and 35° for the inferior surfaces 60. For PAS45 block the angles are 45° for the superior surfaces 55 and 50° for the inferior surfaces 60.

As such, the upper or superior portion 55 of the PAS blocks have bearing elements facing essentially in a medial or inward direction while the lower or inferior facets 60 face laterally or outward. The PAS block is machined, cast, or otherwise formed to have two holes 50 in the superior portion of the block and two holes 50 in the inferior portion to receive the facet bearing buttons 10. These holes are sized to provide a “snap” function for the facet bearing button.

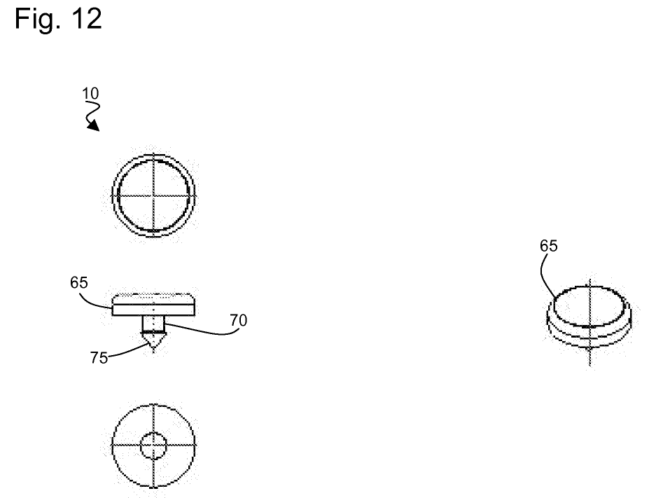

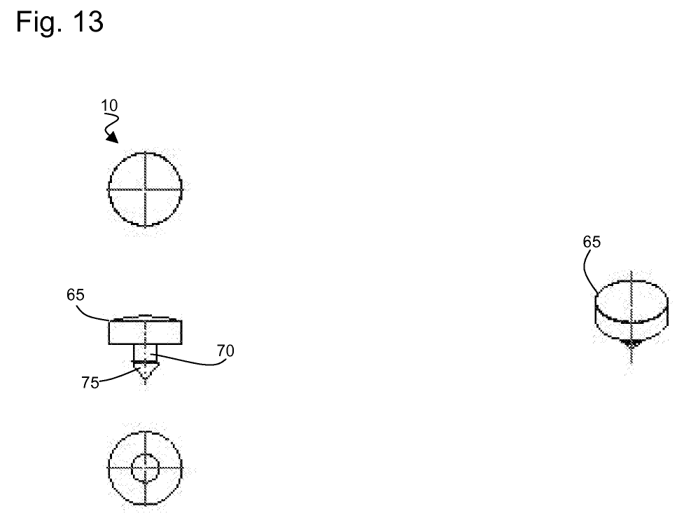

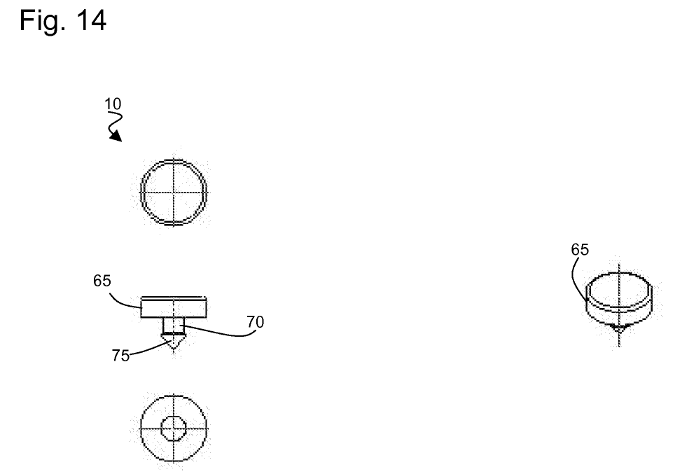

With reference to FIG. 12 through FIG. 15, the facet bearing buttons are fabricated from ultra-high molecular weight polyethylene (UHMWPE) or other biocompatible material. In a particular embodiment, the buttons have a mushroom-like shape 65 with a barb 75 or arrowhead-like shape on the button stem 70. The stem 70 snaps into one of the four holes 50 in the posterior arch support block by virtue of compression of the arrowhead barb. All four holes 50 are adapted to receive a facet bearing button. Various size bearing buttons 10, having similar size stems 70, are fabricated and available for each PAS block 7. This feature allows use of one posterior arch support block 7 size in several vertebra locations with only the wear element buttons 10 selected to fit the device to a specific patient application. The bearing surface of the facet bearing button may be slightly concave, as shown in FIG. 12, slightly convex, as shown in FIG. 13 or flat, as shown in FIG. 14.

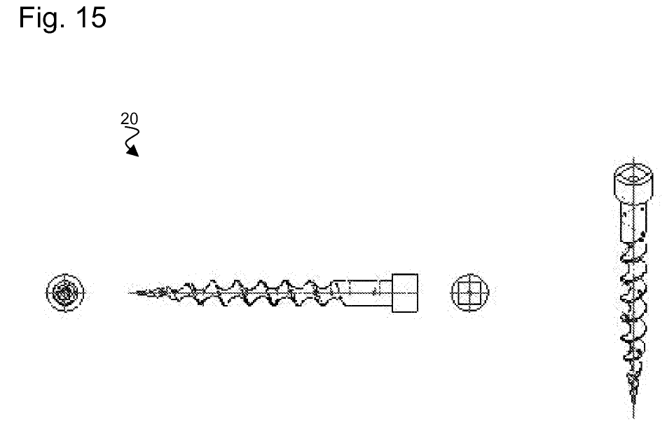

With reference to FIG. 15, the fenestrated screws 20, preferred to be of a resorbable material such as poly lactic and/or poly Glycolic acid polymers, serve to attach the posterior arch structure block 7 to the host vertebra in approximately the same location as where the pedicles were removed. The holes in the screws 20 allow bone to grow through them further incorporating the stems 5 into the host vertebral body. The screws are angled such that they engage through the posterior arch structure 7 into the host vertebral body with the points approximately at the most anterior vertebral region.

A method of maneuvering the prostheses during implantation is necessary because the prostheses morphology is complex and it is larger than the available opening between the lower articular processes of the superior vertebra and the upper articular processes of the inferior vertebra. The maneuver begins with the superior end of the prostheses 1 being placed in first, with the posterior arch structure 7 pedicles 5 facing caudally, then a rotation maneuver about the posterior arch structure lateral axis is performed such that the prosthetic posterior arch structure stems line up with the prepared holes and the superior prosthetic facets appropriately engage the inferior facets of the superior vertebral body. The posterior arch structure stems are then inserted into the prepared holes.

It will be seen that the objects set forth above, and those made apparent from the foregoing description, are efficiently attained and since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention which, as a matter of language, might be said to fall therebetween. Now that the invention has been described,

What is claimed is:

1. A prosthesis for the replacement of at least a portion of the bone of a facet located on a host mammalian vertebra, comprising:
a posterior arch structure having a plurality of bearing attachment surfaces;
a plurality of facet bearing buttons releasably engaged with the bearing attachment surfaces; and
a plurality of bioresorbable attachment screws to secure the posterior arch structure to the posterior vertebral articular process of the mammalian vertebra.

2. The prosthesis of claim 1, wherein the posterior arch structure further comprises, at least one pedicle extending from the posterior arch structure, the at least one pedicle to mate with at least one fenestra of the mammalian vertebra and to receive at least one of the plurality of bioresorbable attachment screws.

3. The prosthesis of claim 1, wherein the pedicles are cylindrical in shape.

4. The prosthesis of claim 1, wherein the posterior arch structure further comprises a posterior protrusion in a shape approximating a spinous process.

5. The prosthesis of claim 4, wherein the posterior protrusion further comprises an attachment location to resect a bone piece originating from the spinous process of the mammalian vertebra.

6. The prosthesis of claim 4, wherein the posterior protrusion is capable of bioincorporation into the spinous process of the mammalian vertebra.

7. The prosthesis of claim 4, wherein the posterior protrusion is fabricated comprising a plurality of volumetric voids to establish volumetric porosity of the posterior protrusion.

8. The prosthesis of claim 7, wherein the volumetric voids establishing the volumetric porosity of the posterior arch structure are between about 75 μm and 500 μm in diameter.

9. The prosthesis of claim 4, wherein the posterior protrusion further comprises at least a portion of osteoinductive materia.

10. The prosthesis of claim 9, wherein the osteoinductive material is hydroxyapitite.

11. The prosthesis of claim 4, wherein the posterior portion further comprises at least a portion of osteoconductive materia.

12. The prosthesis of claim 11, wherein the osteoconductive material is bone morphogenic protein.

13. The prosthesis of claim 1, wherein the plurality of bearing attachment surfaces further comprises, at least one superior prosthetic facet to contact an inferior facet of a superior vertebral body of the vertebra, the at least one superior prosthetic facet to receive at least one of the plurality of facet bearing buttons.

14. The prosthesis of claim 1, wherein the plurality of bearing attachment surfaces further comprises, at least one inferior prosthetic facet to contact a superior facet of an inferior vertebral body of the vertebra, the at least one inferior prosthetic facet to receive at least one of the plurality of facet bearing buttons.

15. The prosthesis of claim 1, wherein the posterior arch structure is fabricated of a material selected from the group consisting of ceramic, glass reinforced polymer and carbon reinforced polymer.

16. The prosthesis of claim 1, wherein the posterior arch structure is capable of bioincorporation into the host vertebra.

17. The prosthesis of claim 1, wherein the posterior arch structure is fabricated comprising a plurality of volumetric voids to establish volumetric porosity of the posterior arch structure.

18. The prosthesis of claim 17, wherein the volumetric voids establishing the volumetric porosity of the posterior arch structure are between about 75 μm and 500 μm in diameter.

19. The prosthesis of claim 1, wherein the posterior arch structure further comprises at least a portion of osteoinductive materia.

20. The prosthesis of claim 19, wherein the osteoinductive material is hydroxyapitite.

21. The prosthesis of claim 1, wherein the posterior arch structure further comprises at least a portion of osteoconductive materia.

22. The prosthesis of claim 21, wherein the osteoconductive material is bone morphogenic protein.

23. The prosthesis of claim 1, wherein the plurality of bearing attachment surfaces are substantially planar and positioned at a predetermined angle with respect to a sagittal plane of the host.

24. The prosthesis of claim 13, wherein the at least one of the superior bearing attachment surfaces is positioned to face substantially medially relative to the posterior arch structure.

25. The prosthesis of claim 13, wherein the at least one of the superior bearing attachment surfaces is positioned at substantially a 30 degree angle relative to the sagittal plane of the host.

26. The prosthesis of claim 13, wherein the at least one of the superior bearing attachment surfaces is positioned at substantially a 45 degree angle relative to the sagittal plane of the host.

27. The prosthesis o claim 13, wherein the at least on of the inferior bearing attachment surfaces is positioned to face substantially laterally relative to the posterior arch structure.

28. The prosthesis of claim 14, wherein the at least one of the inferior bearing attachment surfaces is positioned at substantially a 35 degree angle relative to the sagittal plane of the host.

29. The prosthesis of claim 14, wherein the at least one of the inferior bearing attachment surfaces is positioned at substantially a 50 degree angle relative to the sagittal plane of the host.

30. The prosthesis of claim 1, wherein the plurality of facet bearing buttons are fabricated from ultra-high molecular weight polyethylene.

31. The prosthesis of claim 1, wherein the plurality of facet bearing buttons further comprises a button cap and a button stem, the button stem adapted to allow releasable engagement with an aperture of the plurality of bearing attachment surfaces.

32. The prosthesis of claim 31, wherein the button cap of the facet bearing button is fabricated to have a contour selected from the contours consisting of substantially convex, substantially concave and substantially flat.

33. The prosthesis of claim 31, wherein the button stem further comprises a locking barb to secure the facet bearing button within the aperture of the bearing attachment surface.

34. The prosthesis of claim 1, wherein the bioresorbable attachment screws are fabricated of a polymer material.

35. A prosthesis for the replacement of at least a portion of the bone of a facet located on a host mammalian vertebra, comprising:
a posterior arch structure having two superior bearing attachment surfaces and two inferior bearing attachment surfaces;
two pedicles extending from the posterior arch structure, the two pedicles dimensioned to engage with two fenestra provided in the host vertebra;
two superior facet bearing buttons releasably engaged with the two superior bearing attachment surfaces;
two inferior facet bearing buttons releasably engaged with the two inferior bearing attachment surfaces; and
two bioresorbable attachment screws to secure the posterior arch structure to the posterior vertebral articular process of the mammalian vertebra through the two pedicles.
